(19) United States
(12) Patent Application Publication
(10) Pub. No.: US 2010/0037403 A1
(43) Pub. Date: Feb. 18, 2010

(54) Hair color mini's

(76) Inventor: Mina Houtan, Egg Harbor Township, NJ (US)

Correspondence Address: Mina Houtan, Ph.D., 300 Central Avenue, Egg Harbor Township, NJ, 08234, US

(21) Appl. No.: 12/228,417

(22) Filed: Aug. 13, 2008

(51) Int. Cl. A61K 8/22; A61Q 5/10

(52) U.S. Cl. 8/406

(57) ABSTRACT

The invention relates to an improved method for applying hair colorant which will result in less pollution of the environment and save the user money. A hair colorant packaging system for home use, capable of separately housing two hair colorant solutions in smaller containers which the user can prepare prior to use on the hair—without preparing too much, thus wasting the product. Because of the smaller packages, the user is capable of preparing just the right amount needed.

### BACKGROUND OF THE INVENTION

1. Field of the Invention

This improved invention relates to a method for applying hair colorant with no waste of product and minimizing environmental waste. More specifically, it relates to hair colorant in single use packs to avoid waste when the user requires only touching up the hair at the roots, sideburns or hairline on the frame of the face. Any combination of hair colorant chemical compositions available on the market today can be used within this hair colorant kit for the home.

2. Background Art

Consumers demand high performance hair colorants today, and while there are many variations of hair colorants and application methods for such, they all require the user to mix large quantities of two separate products prior to applying it to one's hair and then recommending that you dispose of the unused product for safety reasons. This result's in waste when the user only intends to touch-up small areas of the hair. Once hair colorant is used, touch-ups are required on a repeated basis to keep the hair color even and minimize the gray appearance. Since consumers purchase hair colorants on a repeated basis to minimize the appearance of gray hair, the waste of product and the disposing of packaging can be phenomenal; not only in dollars and cents, but to the environment. Over one third of the population use hair colorants regularly. Hair colorants were always used in salons and in the home, however the advantage of this invention is that you use only what is needed. No waste, no mess, and it's environmentally friendly. You can also carry it in a travel kit when you need to.

When hair colorant is used just to do touch-ups, the user either has to use the entire bottle of the mixed solution, which can result in the hair color being uneven or the user has to waste the unused solution because once you mix it, you cannot save the unused portion for later use; therefore you must throw it away, wasting money. The methods for application and the chemical composition has been improved time and time again, however this improved invention is directed towards how the hair colorant is packaged and used to prevent waste. It will limit environmental waste and save the user money.

### BRIEF SUMMARY OF THE INVENTION

This improved invention is of a hair colorant kit for home use that has individual, single use packages that get mixed together and applied to the hair as needed with no waste of the product because of the compact packaging. Just like any hair colorant kit for the home, this hair colorant kit will require the user to mix two separate single packages of product, which when combined will create one of any of the available chemical compositions on the market today that would change a person's hair color, with the difference being that this improved hair colorant kit will allow the user to do small areas of hair that only require touch up with the result being little to no waste of product or to the environment.

### BRIEF DESCRIPTION OF THE DRAWINGS

FIG. 1

Is the frontal view of a standard cardboard box which measures 9×18 cm. This box will contain the hair colorant kit which includes a mixing tray, mixing stick, application brush, 12 packs of solution (6 each of the hair colorant composition and 6 each of a peroxide composition), and 2 pairs of non-latex gloves.

FIG. 1a

Is the side view of the same standard cardboard box from FIG. 1 measuring 4 cm.

FIG. 2

Is of the oval, plastic mixing tray contained within the kit, measuring 7×12×3.5 cm showing grooved openings on either side of the tray for resting the mixing and application tools. At it's widest point this tray will be 7 cm and at it's most narrow points it will measure 4 cm.

FIG. 2a

Is a larger diagram of one of the grooved openings, which measure 6 mm on each side of the mixing tray.

FIG. 3

Shows a diagram of the plastic application brush used with the hair colorant kit measuring 14 cm in length with the handle comprising of 10 cm of the total length and the head comprising of 4 cm of the total length and having a scalloped edge. The depth of the bristles and the plastic head combine to measure 10 mm in height.

FIG. 3a

Shows another view of the plastic application brush.

FIG. 3b

Is a larger view of the scalloped edge of the application brush, which is intended to allow the user to have better control while applying the product to the hair.

FIG. 3c

Shows the bristles of the application brush which measure ¼ mm thick with a length of 9 mm and can contain up to 250 bristles per brush.

FIG. 4

Is a view of the package which contains the individual components of hair colorant composition and peroxide composition. These packages are made of a leak proof material such as the packaging for single serve ice tea mixes or coffees. These packages measure 10 mm in width with 12 cm in height.

FIG. 4a

Shows how the package is opened and the product is dispensed.

FIG. 5

Is the mixing stick used in this kit measuring 10 mm wide at the base and 5 mm wide at the top and 12 cm in length having a rounded end such as a spoon for easier mixing and preparation of hair colorant.

FIG. 6

Is a diagram of standard, one size fits all non-latex gloves, included in this kit. This kit will contain 2 pairs of gloves.

FIG. 7

Shows the areas for which this hair colorant kit is intended for use. Identified are the roots of the hair at the top of the head and the hairline on the frame of the face.

### DETAILED DESCRIPTION OF THE INVENTION

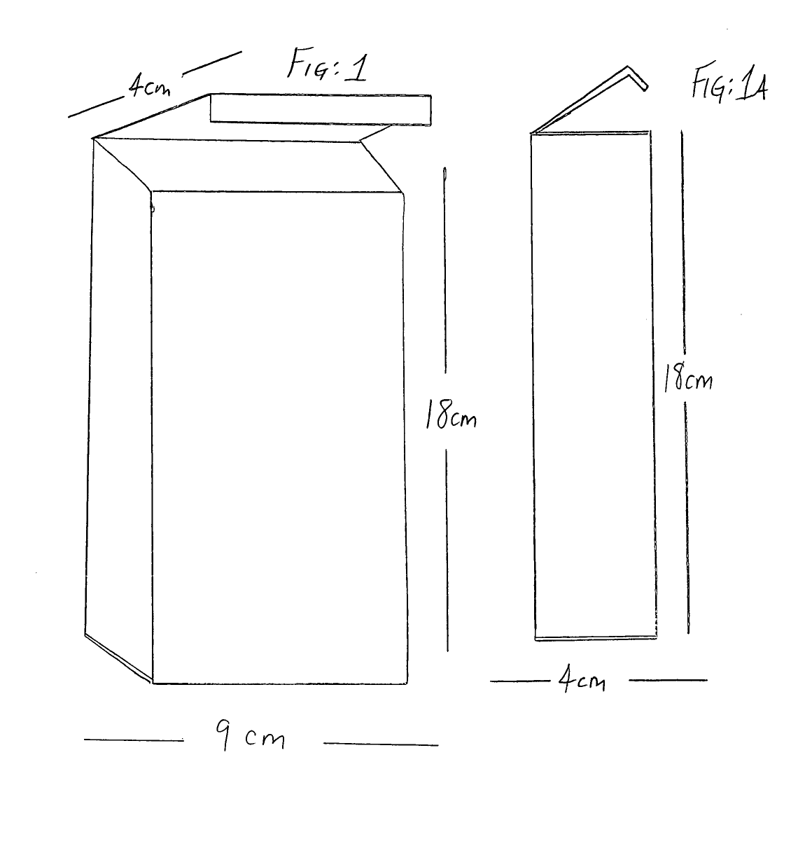

The advantages of this present invention can be more appreciated through the following detailed description. This improved invention is of a less costly method of applying hair colorant to the hair, while minimizing environmental waste. Referring to FIG. 1, this hair colorant kit for the home will comprise of multiple, individual packets of hair colorant composition and peroxide composition, when combined to be used in much smaller quantities because you only need to open and use as little or as much as you need for the area to be treated. The finished composition for the hair colorant can be one of many of the available hair colorants on the market today. This improved invention is only to provide a method for application of hair colorant. This kit will contain everything you need to do hair color touch-ups, without the need to throw out unused product because there won't be any.

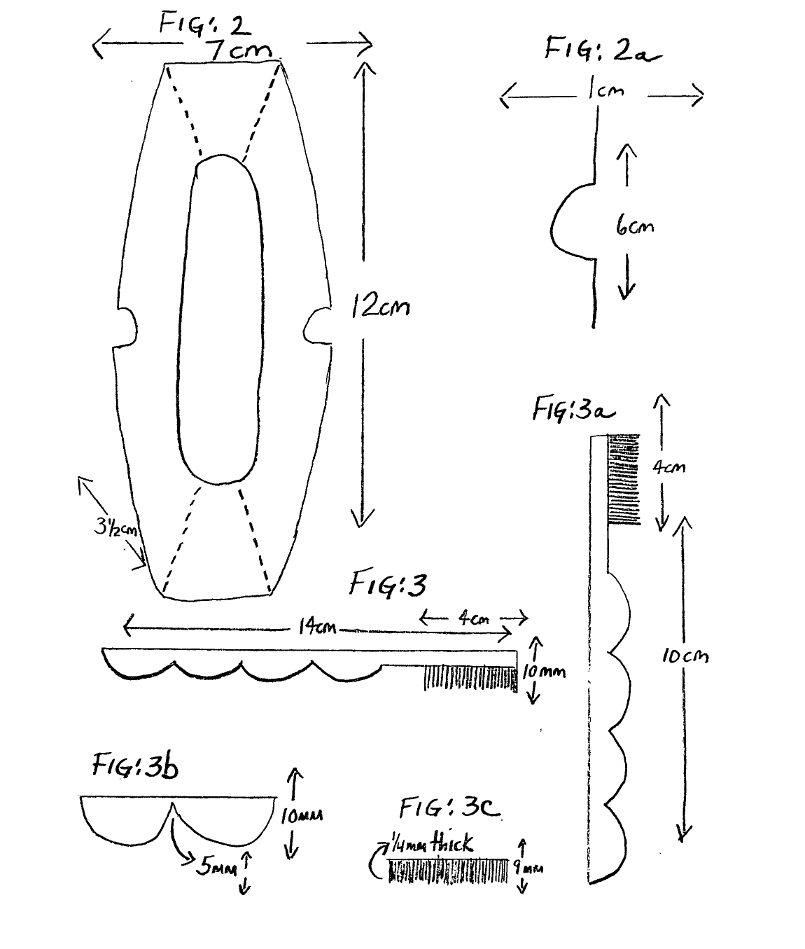

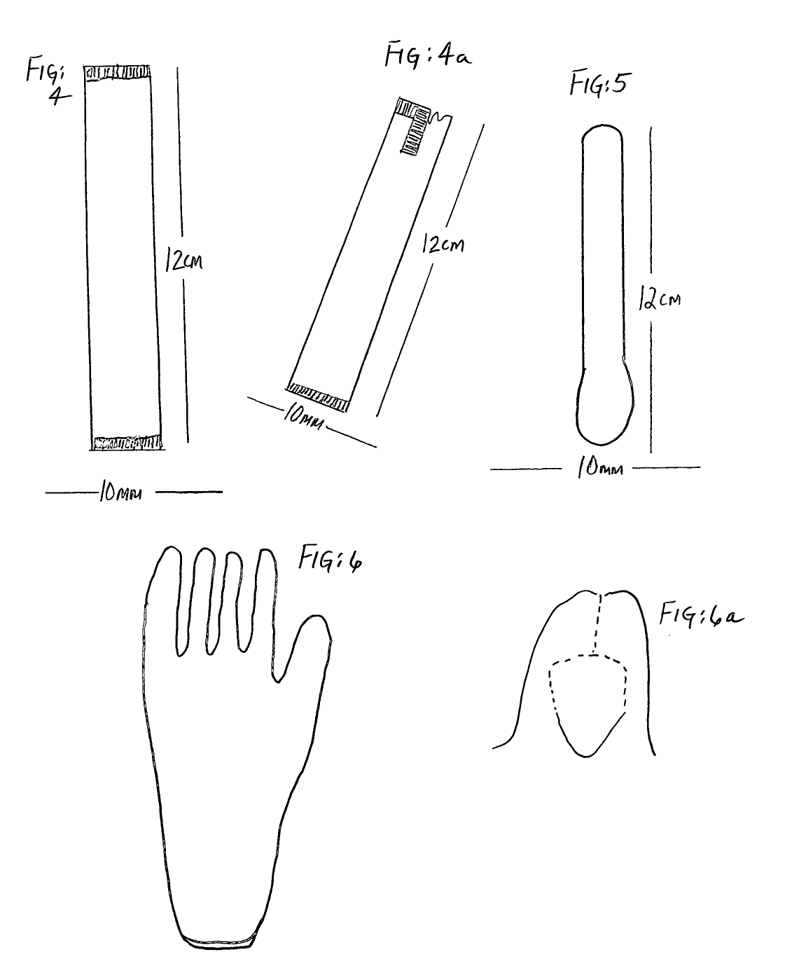

The composition of the hair colorant in this kit should be of a thicker consistency. The application brush as shown in FIG. 3, is similar to a tooth brush, having multiple bristles and a comfortable handle that fits in the user's hand easily—for better control when applying the hair colorant, it is therefore required that the finished solution should be thick—as to adhere to the application brush. The user would first pour 1 of each of the hair colorant composition packets with the peroxide composition packet (FIG. 4), into the plastic, lightweight mixing tray (FIG. 2) and mix well using the mixing stick (FIG. 5)—which can be lightweight plastic or wood, just prior to use on the hair. The gloves in FIG. 6 are of a non-latex material and the kit would bring 2 pairs. With the exception of the individual packets of hair colorant composition and peroxide composition packets, all remaining items are similar to what is already available for use today in hair colorant kits for the home. The designs are slightly varied; therefore those skilled in the art will not find them difficult to make.

Once the product is mixed and ready to use on the hair, using the application brush (FIG. 3), the user would rub the bristle end of the brush into the prepared mixture and simply brush it into the roots or on the hairline as needed (FIG. 6a). The scalloped edge of the application brush (FIG. 3) is designed specifically to fit in-between a person's finger for a better grip on the brush. This can be made of a lightweight, yet durable, re-usable plastic. Because this hair colorant home kit will have multiple uses, the individual pieces to be used such as the mixing tray (FIG. 2), the mixing stick (FIG. 5), and the application brush (FIG. 3) must be washable and re-usable plastic. There are many patents today for hair colorant and application methods, once again this improved invention is of yet another method for preparation and application of hair colorant to the hair.

1. An improved kit comprising of multiple, individual mini applications of hair colorant.

2. The kit of claim 1 wherein the receptacles are small enough to carry in your pocket.

3. A method for applying hair colorant comprising of a scalloped, rubber coated handled brush for easy gripping during application.
